(19) United States
(12) Patent Application Publication
(10) Pub. No.: US 2024/0239893 A1
(43) Pub. Date: Jul. 18, 2024

(54) METHODS AND COMBINATIONS FOR THE TREATMENT OF CANCER USING IMMUNE CHECKPOINT INHIBITOR ANTIBODIES

(71) Applicant: AstraZeneca AB, Sodertalie (SE)

(72) Inventors: John Kurland, Gaithersburg, MD (US); Alejandra Negro, Gaithersburg, MD (US); Shao-Chun Chang, Gaithersburg, MD (US)

(21) Appl. No.: 18/620,387

(22) Filed: Mar. 28, 2024

Related U.S. Application Data
(63) Continuation of application No. 17/318,017, filed on May 12, 2021.
(60) Provisional application No. 63/023,554, filed on May 12, 2020.

(51) Int. Cl. C07K 16/28; A61K 45/06; A61P 35/00; A61K 39/00

(52) U.S. Cl. CPC C07K 16/2818; A61K 45/06; A61P 35/00; C07K 16/2827; A61K 2039/507; A61K 2039/545

(57) ABSTRACT

The disclosure relates to methods, compositions, and combinations for the treatment of cancer. Specifically, the disclosure relates to methods comprising administering to a subject in need thereof at least one of an anti-CTLA-4 antibody or an antigen-binding fragment and an anti-PD-L1 antibody or an antigen-binding fragment thereof. The disclosure also relates to combinations comprising at least one of an anti-CTLA-4 antibody or an antigen-binding fragment and an anti-PD-L1 antibody or an antigen-binding fragment thereof.

This application is a continuation of U.S. patent application Ser. No. 17/318,017, filed May 12, 2021, which claims the benefit of U.S. Provisional Patent Application No. 63/023,554, filed May 12, 2021, the disclosure of which is explicitly incorporated herein in its entirety by reference.

### SEQUENCE LISTING STATEMENT

A computer readable form of the Sequence Listing is filed with this application by electronic submission and is incorporated into this application by reference in its entirety. The Sequence Listing is contained in the file created on Mar. 28, 2024 having the file name “19-992-US-.xml” and is 21,345 bytes in size.

### FIELD OF THE DISCLOSURE

The disclosure relates to methods, compositions, and combinations for the treatment of cancer. Specifically, the disclosure relates to methods comprising administering to a subject in need thereof at least one of an anti-CTLA-4 antibody or an antigen-binding fragment and an anti-PD-L1 antibody or an antigen-binding fragment thereof. The disclosure also relates to combinations comprising at least one of an anti-CTLA-4 antibody or an antigen-binding fragment and an anti-PD-L1 antibody or an antigen-binding fragment thereof.

### BACKGROUND

Hepatocellular Carcinoma (HCC) is the third-leading cause of cancer death worldwide. The current treatment paradigm for HCC utilizes multimodality therapy. For patients with early-stage disease, treatment is based on curative intent, and options include surgical resection, liver transplantation, and/or local regional therapies such as radiofrequency ablation. Unfortunately, those patients who do not qualify for curative treatment are treated with other palliative locoregional therapies, which primarily include bland transarterial embolization (TAE), transarterial embolization with chemotherapy-containing or radioactive particles, or with systemic therapy such as sorafenib, lenvatinib, cabozantinib, and ramucirumab. Additionally, regorafenib may be an option for some advanced patient populations.

As initial treatment for advanced HCC (first line therapy), sorafenib demonstrated improvement in survival, which supported global regulatory approval (Llovet et al., N. Engl. J. Med. 359(4): 378-90 (2008)). Lenvatinib has also been approved as initial treatment for advanced HCC based on non-inferior survival results and improved overall response rate (ORR) and progression-free survival (PFS) compared with sorafenib (Kudo et al., Lancet 391(10126): 1163-73 (2018)). Regorafenib has been shown to moderately extend survival in second-line HCC patients. Despite the variety of new treatment options available, the overall outcome of patients with advanced HCC are still poor. Therefore, HCC represents a significant unmet medical need. The disclosure describes methods, compositions, and combinations for the treatment of HCC that address this unmet medical need.

### SUMMARY

In one aspect, the disclosure herein provides a method of treating a tumor in a subject in need thereof, comprising administering to the subject: (i) a therapeutically effective amount of an anti-PD-L1 antibody or an antigen-binding fragment thereof; and (ii) an anti-CTLA-4 antibody or an antigen-binding fragment thereof at a dose between 1 mg/kg to 10 mg/kg.

In another aspect, the disclosure herein provides a method of treating a tumor in a subject in need thereof, comprising administering to the subject: (i) a therapeutically effective amount of an anti-PD-L1 antibody or an antigen-binding fragment thereof; and (ii) an anti-CTLA-4 antibody or an antigen-binding fragment thereof at a flat dose of between 75 mg to 1120 mg.

In a further aspect, the disclosure herein provides a method of treating a tumor in a subject in need thereof, comprising administering to the subject an anti-CTLA-4 antibody or an antigen-binding fragment thereof at a dose of between 5 mg/kg to 15 mg/kg.

In yet another aspect, the disclosure herein provides a method of treating a tumor in a subject in need thereof, comprising administering to the subject an anti-CTLA-4 antibody or an antigen-binding fragment thereof at a flat dose of between 650 mg to 850 mg.

In another aspect, the disclosure herein provides a combination for the treatment of a tumor in a subject in need thereof, wherein the combination comprises: (i) a therapeutically effective amount of an anti-PD-L1 antibody or an antigen-binding fragment thereof; and (ii) an anti-CTLA-4 antibody or an antigen-binding fragment thereof at a dose between 1 mg/kg to 10 mg/kg.

In a further aspect, the disclosure herein provides a combination for the treatment of a tumor in a subject in need thereof, wherein the combination comprises: (i) a therapeutically effective amount of an anti-PD-L1 antibody or an antigen-binding fragment thereof; and (ii) an anti-CTLA-4 antibody or an antigen-binding fragment thereof at a flat dose of between 75 mg to 1120 mg.

In yet another aspect, the disclosure herein provides a combination for the treatment of a tumor in a subject in need thereof, wherein the combination comprises an anti-CTLA-4 antibody or an antigen-binding fragment thereof at a dose of between 5 mg/kg to 15 mg/kg.

In yet another aspect, the disclosure herein provides a combination for the treatment of a tumor in a subject in need thereof, wherein the combination comprises an anti-CTLA-4 antibody or an antigen-binding fragment thereof at a flat dose of between 650 mg to 850 mg.

### BRIEF DESCRIPTION OF THE DRAWINGS

FIG. 1 illustrates the study flow diagram of treatment using durvalumab and tremelimumab alone and in combination as disclosed in Example 1.

FIG. 2 illustrates the dosing schema of the HIMALAYA study as described disclosed in Example 1.

FIG. 3 illustrates the overall design of the study disclosed in Example 2.

FIG. 4 shows the dosing schema of the study disclosed in Example 2.

FIG. 5 shows the overall survival (OS) rates for each arm of the study disclosed in Example 2.

FIG. 6 shows the OS, survival rate and total treatment duration for patients treated in the study disclosed in Example 2.

FIG. 7A-7D shows that responses for the study disclosed in Example 2 were observed regardless of PD-L1 expression level or viral status. FIG. 7A shows the response for combination therapy with durvalumab 1500 mg plus tremelimumab 300 mg (T300+D). FIG. 7B shows the response for 1500 mg durvalumab monotherapy (D). FIG. 7C shows the response for tremelimumab 750 mg monotherapy (T). FIG. 7D shows the response for combination therapy with durvalumab 1500 mg plus tremelimumab 75 mg (T75+D).

FIG. 8 shows the secondary endpoints measured in the study disclosed in Example 2.

FIG. 9A-9B shows the results of pharmacodynamic biomarker analysis of the patient populations disclosed in Example 2. FIG. 9A shows canon-2 scores observed with T300+D. FIG. 9B shows median counts observed with T300+D.

FIG. 10A-10D shows the best response for target lesion from baseline for the study disclosed in Example 2. FIG. 10A shows Durvalumab. FIG. 10B shows Tremelimumab. FIG. 10C shows T75+D. FIG. 10D shows time since therapy initation.

FIG. 11 shows Kaplan-Meier analysis of PFS for the study disclosed in Example 2.

FIG. 12A-12H shows the correlation (correlation coefficient >0.1) of lymphocyte populations counts with canon-1 or canon-2 scores for the study disclosed in Example 2. FIG. 12A shows CD4+Ki67+. FIG. 12B shows CD4+ICOS+. FIG. 12C shows CD4+HLA-DR+. FIG. 12D shows CD4+Tem. FIG. 12E shows CD8+Ki67+. FIG. 12F shows CD8+ICOS+. FIG. 12G shows CD4+Tcm. FIG. 12H shows CD4+CD38+.

FIG. 13 shows CD3+ CD8+ Ki67+ T cell analysis of patient samples by response at day 1 and day 15 for the study disclosed in Example 2.

FIG. 14A-14B shows there were no significant differences in baseline richness or Simpson clonality of T-cells across arms in the study disclosed in Example 2. FIG. 14A shows Baseline Richness. FIG. 14B shows Baseline Simpson Clonality.

FIG. 15A-15E shows that greater clonal expansion of T cells was associated with response and driven by higher doses of tremelimumab (T). FIG. 15A shows T-cell clones from Day 1-Day 29. FIG. 15B shows study 22: D arm. FIG. 15C shows study 22: T75D arm. FIG. 15D shows study 22: T300D arm. FIG. 15E shows study 22: T10 arm.

FIG. 16A-16E shows that greater clonal expansion of T-cells was associated with better OS and was seen in durvalumab+tremelimumab (D+T) combination arms in the study disclosed in Example 2. FIG. 16A shows results of all study 22 arms combined. FIG. 16B shows results of the D arm. FIG. 16C shows results of the T75D arm. FIG. 16D shows results of the T300D arm. FIG. 16E shows results of the T10 arm.

### DETAILED DESCRIPTION

The disclosure relates to methods, compositions, and combinations for the treatment of cancer. Specifically, the disclosure relates to methods comprising administering to a subject in need thereof at least one of an anti-CTLA-4 antibody or an antigen-binding fragment and an anti-PD-L1 antibody or an antigen-binding fragment thereof. The disclosure also relates to combinations comprising at least one of an anti-CTLA-4 antibody or an antigen-binding fragment and an anti-PD-L1 antibody or an antigen-binding fragment thereof.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

In some embodiments, provided herein is a method of treating a tumor in a subject in need thereof, comprising administering to the subject a therapeutically effective amount of an anti-PD-L1 antibody or an antigen-binding fragment thereof and an anti-CTLA-4 antibody or an antigen-binding fragment thereof at a dose between 1 mg/kg to 5 mg/kg.

In some embodiments, provided herein is a method of treating tumor in a subject in need thereof, comprising administering to the subject a therapeutically effective amount of an anti-PD-L1 antibody or an antigen-binding fragment thereof and an anti-CTLA-4 antibody or an antigen-binding fragment thereof at a flat dose of between 75 mg to 1120 mg.

In some embodiments, provided herein is a method of treating tumor in a subject in need thereof, comprising administering to the subject an anti-CTLA-4 antibody or an antigen-binding fragment thereof at a dose of between 5 mg/kg to 15 mg/kg.

In some embodiments, provided herein is a combination for the treatment of a tumor in a subject in need thereof, wherein the combination comprises a therapeutically effective amount of an anti-PD-L1 antibody or an antigen-binding fragment thereof and an anti-CTLA-4 antibody or an antigen-binding fragment thereof at a dose between 1 mg/kg to 5 mg/kg.

In some embodiments, provided herein is a combination for the treatment of a tumor in a subject in need thereof, wherein the combination comprises a therapeutically effective amount of an anti-PD-L1 antibody or an antigen-binding fragment thereof and an anti-CTLA-4 antibody or an antigen-binding fragment thereof at a flat dose of between 75 mg to 1120 mg.

In some embodiments, provided herein is a combination for the treatment of a tumor in a subject in need thereof, wherein the combination comprises an anti-CTLA-4 antibody or an antigen-binding fragment thereof at a dose of between 5 mg/kg to 15 mg/kg.

The term “antibody,” as used herein, refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “antigen-binding fragment” refers to a portion of an intact antibody and/or refers to the antigenic determining variable domains of an intact antibody. It is known that the antigen-binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

In some embodiments, the anti-PD-L1 antibody or antigen-binding fragment thereof is durvalumab. Durvalumab (MEDI4736, Imfinzi®) is a human monoclonal antibody directed against human PD-L1 that is capable of blocking the binding of PD-L1 to both the PD1 and CD80 receptors. Disclosure related to durvalumab can be found in U.S. Pat. Nos. 8,779,108 and 9,493,565, which are incorporated herein by reference.

Durvalumab and antigen-binding fragments thereof for use in the methods, compositions, and combinations provided herein comprises a heavy chain and a light chain or a heavy chain variable region and a light chain variable region. In some embodiments, durvalumab or antigen-binding fragment thereof for use in the methods, compositions, and combinations provided herein comprises a light chain variable region comprising the amino acid sequence of SEQ ID NO: 1 and a heavy chain variable region comprising the amino acid sequence of SEQ ID NO: 2. In some embodiments, durvalumab or antigen-binding fragment thereof for use in the methods, compositions, and combinations provided herein comprises a heavy chain variable region and a light chain variable region, wherein the heavy chain variable region comprises the Kabat-defined CDR1, CDR2, and CDR3 sequences of SEQ ID NOs: 3-5, and wherein the light chain variable region comprises the Kabat-defined CDR1, CDR2, and CDR3 sequences of SEQ ID NOs: 6-8. Those of ordinary skill in the art would easily be able to identify Chothia-defined, Abm-defined, or other CDR definitions known to those of ordinary skill in the art. In some embodiments, durvalumab or antigen-binding fragment thereof for use in the methods, compositions, and combinations provided herein comprises the variable heavy chain and variable light chain CDR sequences of the 2.14H9OPT antibody as disclosed in U.S. Pat. Nos. 8,779,108 and 9,493,565, which are incorporated herein by reference in their entirety.

Durvalumab light chain (LC) variable region:
(SEQ ID NO: 1)
EIVLTQSPGTLSLSPGERATLSCRASQRVSSSYLAWYQQKPGQAPRLLIYD
ASSRATGIPDRFSGSGSGTDFTLTISRLEPEDFAVYYCQQYGSLPWTFGQG
TKVEIK
Durvalumab heavy chain (HC) variable region:
(SEQ ID NO: 2)
EVQLVESGGGLVQPGGSLRLSCAASGFTFSRYWMSWVRQAPGKGLEWVANI
KQDGSEKYYVDSVKGRFTISRDNAKNSLYLQMNSLRAEDTAVYYCAREGGW
FGELAFDYWGQGTLVTVSS
Durvalumab heavy chain CDRs:
HC-CDR1:
(SEQ ID NO: 3)
GFTFSRYWMS
HC-CDR2:
(SEQ ID NO: 4)
NIKQDGSEKYYVDSVKG
HC-CDR3:
SEQ ID NO: 5)
EGGWFGELAFDY
Durvalumab, light chain CDRs:
LC-CDR1:
(SEQ ID NO: 6)
RASQRVSSSYLA
LC-CDR2:
(SEQ ID NO: 7)
DASSRAT
LC-CDR3:
(SEQ ID NO: 8)
QQYGSLPWT

In some embodiments, the anti-CTLA-4 antibody or antigen-binding fragment thereof is tremelimumab. Tremelimumab and antigen-binding fragments thereof for use in the methods compositions, and combinations provided herein comprises a heavy chain and a light chain or a heavy chain variable region and a light chain variable region. In some embodiments, tremelimumab or antigen-binding fragment thereof for use in the methods compositions, and combinations provided herein comprises a light chain variable region comprising the amino acid sequence of SEQ ID NO: 9 and a heavy chain variable region comprising the amino acid sequence of SEQ ID NO: 10. In some embodiments, tremelimumab or antigen-binding fragment thereof for use in the methods compositions, and combinations provided herein comprises a heavy chain variable region and a light chain variable region, wherein the heavy chain variable region comprises the Kabat-defined CDR1, CDR2, and CDR3 sequences of SEQ ID NOs: 11-13, and wherein the light chain variable region comprises the Kabat-defined CDR1, CDR2, and CDR3 sequences of SEQ ID NOs: 14-16. Those of ordinary skill in the art would easily be able to identify Chothia-defined, Abm-defined, or other CDR definitions known to those of ordinary skill in the art. In some embodiments, tremelimumab or antigen-binding fragment thereof for use in the methods compositions, and combinations provided herein comprises or the variable heavy chain and variable light chain CDR sequences of the 11.2.1 antibody as disclosed in U.S. Pat. No. 6,682,736, which is incorporated herein by reference in its entirety.

Tremelimumab light chain (LC) variable region:
(SEQ ID NO: 9)
PSSLSASVGDRVTITCRASQSINSYLDWYQQKPGKAPKLLIYAASSLQSGV
PSRFSGSGSGTDFTLTISSLQPEDFATYYCQQYYSTPFTFGPGTKVEIKRT
VAAPSVFIFPPSDEQLKSGTASVVCLLNNFYPREAKV
Tremelimumab heavy chain (HC) variable region:
(SEQ ID NO: 10)
GVVQPGRSLRLSCAASGFTFSSYGMHWVRQAPGKGLEWVAVIWYDGSNKY
YADSVKGRFTISRDNSKNTLYLQMNSLRAEDTAVYYCARDPRGATLYYYYY
GMDVWGQGTTVTVSSASTKGPSVFPLAPCSRSTSESTAALGCLVKDYFPEP
VTVSWNSGALTSGVH
Tremelimumab heavy chain CDRs
HC-CDR1:
(SEQ ID NO: 11)
GFTFSSYGMH
HC-CDR2:
(SEQ ID NO: 12)
VIWYDGSNKYYADSV
HC-CDR3:
(SEQ ID NO: 13)
DPRGATLYYYYYGMDV
Tremelimumab light chain CDR1s
LC-CDR1:
(SEQ ID NO: 14)
RASQSINSYLD
LC-CDR2:
(SEQ ID NO: 15)
AASSLQS
LC-CDR3:
(SEQ ID NO: 16)
QQYYSTPFT

As used herein, the term “vascular endothelial growth factor (VEGF) inhibitors” means agents that inhibit the activity of VEGF and VEGFR. VEGR and VEGFR (a tyrosine kinase receptor) signaling modulates angiogenesis, which involves making of new blood vessels from existing blood vessels. Abnormal angiogenesis is known to occur in cancer, degenerative eye conditions, and other conditions that involve inflammation. Specific monoclonal antibodies can be used as VEGF inhibitors and particular tyrosine kinase inhibitors are used as VEGFR inhibitors. Vascular endothelial growth factor (VEGF)/vascular endothelial growth factor receptor (VEGFR) inhibitors are used to treat various types of cancers.

The term “subject” is intended to include human and non-human animals, particularly mammals. In certain embodiments, the subject is a human patient.

In some embodiments, the methods, compositions, and combinations disclosed herein relate to treating a subject for a tumor disorder and/or a cancer disorder. In some embodiments, the tumor is a solid tumor. In some embodiments, the cancer is selected hepatocellular carcinoma (HCC), cholangiocarcinoma or biliary tract cancer, urothelial bladder carcinoma (UBC) or gastric cancer.

The term “solid tumor,” as used herein, refers to an abnormal mass of tissue that normally does not contain cysts or liquid areas.

The terms “treatment” or “treat,” as used herein, refer to both therapeutic treatment and prophylactic or preventative measures. Those in need of treatment include subjects having cancer as well as those prone to having cancer or those in cancer is to be prevented. In some embodiments, the methods, compositions, and combinations disclosed herein can be used for the treatment of cancer. In other embodiments, those in need of treatment include subjects having a tumor as well as those prone to have a tumor or those in which a tumor is to be prevented. In certain embodiments, the methods, compositions, and combinations disclosed herein can be used for the treatment of tumors. In other embodiments, treatment of a tumor includes inhibiting tumor growth, promoting tumor reduction, or both inhibiting tumor growth and promoting tumor reduction.

The terms “administration” or “administering,” as used herein, refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

Provided herein are methods of treating a tumor in a subject in need thereof, comprising administering to the subject an anti-CTLA-4 antibody or an antigen-binding fragment thereof at a dose of between 5 mg/kg to 15 mg/kg. In some embodiments, provided herein is a method of treating tumor in a subject in need thereof, comprising administering to the subject an anti-CTLA-4 antibody or an antigen-binding fragment thereof at a flat dose of between 650 mg to 850 mg.

Also provided herein are combinations for the treatment of a tumor in a subject in need thereof, wherein the combination comprises an anti-CTLA-4 antibody or an antigen-binding fragment thereof at a dose of between 5 mg/kg to 15 mg/kg. In some embodiments, provided herein are combinations for the treatment of a tumor in a subject in need thereof, wherein the combinations comprise an anti-CTLA-4 antibody or an antigen-binding fragment thereof at a flat dose of between 650 mg to 850 mg.

The dose of the anti-CTLA-4 antibody or antigen-binding fragment thereof to be administered to the subject will vary depending, in part, upon the size (body weight, body surface, or organ size) and condition (the age and general health) of the subject.

In particular embodiments, the subject is administered one or more doses of the anti-CTLA-4 antibody or antigen-binding fragment thereof, wherein the dose is 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, or 15 mg/kg. In some embodiments, the subject is administered one or more doses of the anti-CTLA-4 antibody or antigen-binding fragment thereof wherein the dose is 10 mg/kg.

In particular embodiments, the subject is administered one or more flat doses of the anti-CTLA-4 antibody or antigen-binding fragment thereof, wherein the dose is 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1025 mg, 1050 mg, 1075 mg, 1100 mg, or 1120 mg. In some embodiments, the subject is administered one or more flat doses of the anti-CTLA-4 antibody or antigen-binding fragment thereof wherein the dose is 750 mg.

In particular embodiments, a subject presenting with a tumor is administered an anti-CTLA-4 antibody or an antigen-binding fragment thereof only once or infrequently while still providing benefit to the subject. In other embodiments, the subject is administered additional follow-on doses. Follow-on doses can be administered at various time intervals depending on the subject's age, weight, clinical assessment, tumor burden, and/or other factors, including the judgment of the attending physician.

In particular embodiments, the anti-CTLA-4 antibody or antigen-binding fragment thereof is administered over a two-week treatment period, over a four-week treatment period, over a six-week treatment period, over an eight-week treatment period, over a twelve-week treatment period, over a twenty-four-week treatment period, or over a one-year or more treatment period. In certain embodiments, the anti-CTLA-4 antibody or antigen-binding fragment thereof is administered over a three-week treatment period, over a six-week treatment period, over a nine-week treatment period, over a twelve-week treatment period, over a twenty-four-week treatment period, or over a one-year or more treatment period. In certain embodiments, the anti-CTLA-4 antibody or antigen-binding fragment thereof is administered over a two-month treatment period, over a four-month treatment period, or over a six-month or more treatment period.

In particular embodiments, the anti-CTLA-4 antibody or antigen-binding fragment thereof is administered every week, every two weeks, every four weeks, every six weeks, every eight weeks, every ten weeks, or every twelve weeks.

In particular embodiments, provided herein are methods, compositions, and combinations for the treatment a tumor in a subject in need thereof, wherein tremelimumab is administered to the subject at a dose of 10 mg/kg every four weeks for seven doses followed by the administration to the subject of tremelimumab at a dose of 10 mg/kg every twelve weeks. In particular embodiments, provided herein are methods, compositions, and combinations for the treatment of a tumor in a subject in need thereof, wherein tremelimumab is administered to the subject at a dose of 750 mg every four weeks for seven doses followed by the administration to the subject of tremelimumab at a dose of 750 mg every twelve weeks.

The terms “co-administered,” “in combination,” or “combination therapy,” as used herein, refer to simultaneous or sequential administration of multiple compounds or agents. A first compound or agent may be administered before, concurrently with, or after administration of a second compound or agent. The first compound or agent and the second compound or agent may be simultaneously or sequentially administered on the same day, or may be sequentially administered within 1 day, 2 days, 3 days, 4 days, 5 days, 6 days, 1 week, 2 weeks, 3 weeks, or 1 month of each other. In some embodiments, compounds or agents are co-administered during the period in which each of the compounds or agents are exerting at least some physiological effect and/or has remaining efficacy.

In some embodiments, the anti-CTLA-4 antibody or antigen-binding fragment thereof can be administered in combination with an anti-PD-L1 antibody or an antigen-binding fragment thereof.

The combination therapy dose of the anti-CTLA-4 antibody or antigen-binding fragment thereof with an anti-PD-L1 antibody or an antigen-binding fragment thereof will vary depending, in part, upon the size (body weight, body surface, or organ size) and condition (the age and general health) of the subject. In particular embodiments, the subject is administered one or more doses of the anti-CTLA-4 or antigen-binding fragment thereof as a combination therapy wherein the dose is 1 mg/kg, 2 mg/kg, 3 mg/kg, 4 mg/kg, or 5 mg/kg. In particular embodiments, the subject is administered one or more flat doses of the anti-CTLA-4 antibody or antigen-binding fragment thereof, wherein the dose is 75 mg, 100 mg, 125 mg, 150 mg, 175 mg, 200 mg, 225 mg, 250 mg, 275 mg, 300 mg. 325 mg, 350 mg, 375 mg, 400 mg, 425 mg, 450 mg, 475 mg, or 500 mg.

The combination therapy dose of the anti-PD-L1 antibody or antigen-binding fragment thereof will vary depending, in part, upon the size (body weight, body surface, or organ size) and condition (the age and general health) of the subject. In particular embodiments, the subject is administered one or more doses of the anti-PD-L1 antibody or antigen-binding fragment thereof as a combination therapy wherein the dose is 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, or 25 mg/kg. In particular embodiments, the subject is administered one or more flat doses of the anti-PD-L1 antibody or antigen-binding fragment thereof, wherein the dose is 1110 mg, 1125 mg, 1150 mg, 1175 mg, 1200 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, or 1600 mg.

In particular embodiments, a subject presenting with a tumor is administered a combination therapy comprising an anti-CTLA-4 antibody or an antigen-binding fragment thereof and an anti-PD-L1 antibody or an antigen-binding fragment thereof only once or infrequently while still providing benefit to the subject. In further embodiments, the subject is administered additional follow-on doses. Follow-on doses can be administered at various time intervals depending on the subject's age, weight, clinical assessment, tumor burden, and/or other factors, including the judgment of the attending physician.

In particular embodiments, a subject is administered a combination therapy comprising an anti-CTLA-4 antibody or an antigen-binding fragment and an anti-PD-L1 antibody or an antigen-binding fragment thereof over a two-week treatment period, over a four-week treatment period, over a six-week treatment period, over an eight-week treatment period, over a twelve-week treatment period, over a twenty-four-week treatment period, or over a one-year or more treatment period. In particular embodiments, a subject is administered a combination therapy comprising an anti-CTLA-4 antibody or an antigen-binding fragment and an anti-PD-L1 antibody or an antigen-binding fragment thereof over a three-week treatment period, over a six-week treatment period, over a nine-week treatment period, over a twelve-week treatment period, over a twenty-four-week treatment period, or over a one-year or more treatment period.

In particular embodiments, a subject is administered a combination therapy comprising an anti-CTLA-4 antibody or an antigen-binding fragment and an anti-PD-L1 antibody or an antigen-binding fragment thereof every two weeks, every four weeks, every six weeks, every eight weeks, every ten weeks, or every twelve weeks.

In particular embodiments, the anti-PD-L1 antibody or antigen-binding fragment thereof and the anti-CTLA-4 antibody or antigen-binding fragment thereof are administered simultaneously, separately, or sequentially.

In particular embodiments, a subject is administered a combination therapy comprising an anti-CTLA-4 antibody or an antigen-binding fragment and an anti-PD-L1 antibody or an antigen-binding fragment thereof four times followed by administration of the anti-PD-L1 antibody or an antigen-binding fragment thereof every four weeks.

In particular embodiments, a subject is administered a combination therapy comprising an anti-PD-L1 antibody or an antigen-binding fragment thereof and the anti-CTLA-4 antibody or the antigen-binding fragment for one dose followed by administration of the anti-PD-L1 antibody or an antigen-binding fragment thereof every four weeks.

In particular embodiments, the is administered a combination therapy comprising an anti-PD-L1 antibody or an antigen-binding fragment thereof that is durvalumab administered at a dose of 20 mg/kg and an anti-CTLA-4 antibody or an antigen-binding fragment thereof that is tremelimumab administered at a dose of 1 mg/kg.

In particular embodiments, the subject is administered a combination therapy comprising an anti-PD-L1 antibody or an antigen-binding fragment thereof that is durvalumab at a dose of 1500 mg and an anti-CTLA-4 antibody or an antigen-binding fragment thereof that is tremelimumab at a dose of 75 mg.

In particular embodiments, the subject is administered a combination therapy comprising an anti-PD-L1 antibody or an antigen-binding fragment thereof that is durvalumab at a dose of 1500 mg and an anti-CTLA-4 antibody or an antigen-binding fragment thereof that is tremelimumab at a dose of 300 mg.

In certain embodiments, combination treatment further includes administration of a VEGFR tyrosine kinase inhibitor (TKI) including, but not limited to: ziv-aflibercept, bevacizumab, pazopanib, sunitinib, sorafenib, lenvatinib, cabozantinib, regorafenib, ponatinib, ramucirumab, and vandetanib. In certain embodiments, combination treatment further includes administration of an anti-TIGIT antibody, monalizumab, oleanolic acid, and/or oleclumab.

The methods, compositions, and combinations disclosed herein can be further combined with conventional cancer therapies such as bland transarterial embolization (TAE), transarterial embolization with chemotherapy-containing or radioactive particles chemotherapy, radiation therapy, thermotherapy, surgery (tumor resection), and TACE (transarterial chemoembolization), to treat subjects suffering from tumors or harboring cancer cells. In particular embodiments, methods, compositions, and combinations further comprise administering TACE to the subject.

The terms “pharmaceutical composition” or “therapeutic composition,” as used herein, refer to a compound or composition capable of inducing a desired therapeutic effect when properly administered to a subject. In some embodiments, the disclosure provides a pharmaceutical composition comprising a pharmaceutically acceptable carrier and a therapeutically effective amount of at least one antibody of the disclosure.

The terms “pharmaceutically acceptable carrier” or “physiologically acceptable carrier,” as used herein, refer to one or more formulation materials suitable for accomplishing or enhancing the delivery of one or more antibodies of the disclosure.

When used for in vivo administration, the formulations of the disclosure should be sterile. The formulations of the disclosure may be sterilized by various sterilization methods, including, for example, sterile filtration or radiation. In one embodiment, the formulation is filter sterilized with a presterilized 0.22-micron filter. Sterile compositions for injection can be formulated according to conventional pharmaceutical practice as described in “Remington: The Science & Practice of Pharmacy,” 21st ed., Lippincott Williams & Wilkins, (2005).

In some embodiments, antibodies can be formulated for particular routes of administration, such as oral, nasal, pulmonary, topical (including buccal and sublingual), rectal, vaginal, and/or parenteral administration. The terms “parenteral administration” and “administered parenterally,” as used herein, refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion. Formulations of the disclosure that are suitable for topical or transdermal administration include powders, sprays, ointments, pastes, creams, lotions, gels, solutions, patches, and inhalants. The antibodies and other actives may be mixed under sterile conditions with a pharmaceutically acceptable carrier, and with any preservatives, buffers, or propellants which may be required (see, e.g., U.S. Pat. Nos. 7,378,110; 7,258,873; and 7,135,180; U.S. Patent Application Publication Nos. 2004/0042972 and 2004/0042971).

The formulations can be presented in unit dosage form and can be prepared by any method known in the art of pharmacy. Actual dosage levels of the active ingredients in the formulation of the present disclosure may be varied so as to obtain an amount of the active ingredient which is effective to achieve the desired therapeutic response for a particular subject, composition, and mode of administration, without being toxic to the subject (e.g., “a therapeutically effective amount”). Dosages can also be administered via continuous infusion (such as through a pump). The administered dose may also depend on the route of administration. For example, subcutaneous administration may require a higher dosage than intravenous administration.

Without limiting the disclosure, a number of embodiments of the disclosure are described herein for purpose of illustration.

Item 1. A method of treating a tumor in a subject in need thereof, comprising administering to the subject: (i) a therapeutically effective amount of an anti-PD-L1 antibody or an antigen-binding fragment thereof; and (ii) an anti-CTLA-4 antibody or an antigen-binding fragment thereof at a dose between 1 mg/kg to 10 mg/kg.

Item 2. The method according to item 1, wherein the anti-CTLA-4 antibody or antigen-binding fragment thereof is administered at a dose of 1 mg/kg.

Item 3. The method according to either item 1 or 2, wherein the anti-PD-L1 antibody or antigen-binding fragment thereof is administered at a dose of between 15 mg/kg to 25 mg/kg.

Item 4. The method according to any one of items 1 to 3, wherein the anti-CTLA-4 antibody or antigen-binding fragment thereof is tremelimumab.

Item 5. The method according to any one of items 1 to 4, wherein the anti-PD-L1 antibody or antigen-binding fragment thereof is durvalumab.

Item 6. The method according to any one of items 1 to 5, wherein the anti-PD-L1 antibody or antigen-binding fragment thereof and the anti-CTLA-4 antibody or antigen-binding fragment thereof are administered every four weeks.

Item 7. The method according to item 6, wherein the anti-PD-L1 antibody or antigen-binding fragment thereof and the anti-CTLA-4 antibody or antigen-binding fragment thereof are administered every four weeks for four doses followed by administration of the anti-PD-L1 antibody or antigen-binding fragment thereof every four weeks.

Item 8. The method according to any one of items 1 to 7, wherein the anti-PD-L1 antibody or antigen-binding fragment thereof and the anti-CTLA-4 antibody or antigen-binding fragment thereof are administered simultaneously, separately, or sequentially.

Item 9. The method according to any one of items 1 to 8, further comprising administering transarterial chemoembolization (TACE).

Item 10. The method according to any one of items 1 to 9, wherein the tumor is a hepatocellular carcinoma (HCC).

Item 11. The method according to any one of items 1 to 10, wherein the anti-PD-L1 antibody or antigen-binding fragment thereof is durvalumab administered at a dose of 20 mg/kg and the anti-CTLA-4 antibody or antigen-binding fragment thereof is tremelimumab administered at a dose of 1 mg/kg.

Item 12. A method of treating a tumor in a subject in need thereof, comprising administering to the subject: (i) a therapeutically effective amount of an anti-PD-L1 antibody or an antigen-binding fragment thereof; and (ii) an anti-CTLA-4 antibody or an antigen-binding fragment thereof at a flat dose of between 75 mg to 1120 mg.

Item 13. The method according to item 12, wherein the anti-PD-L1 antibody or antigen-binding fragment thereof is administered at a dose of between 1000 mg to 1600 mg.

Item 14. The method according to item 12, wherein the anti-CTLA-4 antibody or antigen-binding fragment thereof is administered at a dose of 75 mg.

Item 15. The method according to item 12, wherein the anti-CTLA-4 antibody or antigen-binding fragment thereof is administered at a dose of 300 mg.

Item 16. The method according to any one of items 12 to 15, wherein the anti-CTLA-4 antibody or antigen-binding fragment thereof is tremelimumab.

Item 17. The method according to any one of items 12 to 16, wherein the anti-PD-L1 antibody or antigen-binding fragment thereof is durvalumab.

Item 18. The method according to any one of items 12 to 17, wherein the anti-PD-L1 antibody or antigen-binding fragment thereof and the anti-CTLA-4 antibody or the antigen-binding fragment thereof are administered every four weeks.

Item 19. The method according to item 18, wherein the anti-PD-L1 antibody or antigen-binding fragment thereof and the anti-CTLA-4 antibody or antigen-binding fragment thereof are administered every four weeks for four doses followed by administration of the anti-PD-L1 antibody or antigen-binding fragment thereof every four weeks.

Item 20. The method according to any one of items 12 to 19, wherein the anti-PD-L1 antibody or antigen-binding fragment thereof and the anti-CTLA-4 antibody or the antigen-binding fragment thereof are administered for one dose followed by administration of the anti-PD-L1 antibody or antigen-binding fragment thereof every four weeks.

Item 21. The method according to any one of items 12 to 20, wherein the anti-PD-L1 antibody or antigen-binding fragment thereof and the anti-CTLA-4 antibody or antigen-binding fragment thereof are administered simultaneously, separately, or sequentially.

Item 22. The method according to any one of items 12 to 21, further comprising administering transarterial chemoembolization (TACE).

Item 23. The method according to any one of items 12 to 22, wherein the tumor is a hepatocellular carcinoma (HCC).

Item 24. The method according to any one of items 12 to 23, wherein the anti-PD-L1 antibody or antigen-binding fragment thereof is durvalumab administered at a dose of 1500 mg and the anti-CTLA-4 antibody or antigen-binding fragment thereof is tremelimumab administered at a dose of 75 mg.

Item 25. The method according to any one of items 13 to 25, wherein the anti-PD-L1 antibody or antigen-binding fragment thereof is durvalumab administered at a dose of 1500 mg and the anti-CTLA-4 antibody or antigen-binding fragment thereof is tremelimumab administered at a dose of 300 mg.

Item 26. A method of treating a tumor in a subject in need thereof, comprising administering to the subject an anti-CTLA-4 antibody or an antigen-binding fragment thereof at a dose of between 5 mg/kg to 15 mg/kg.

Item 27. A method of treating a tumor in a subject in need thereof, comprising administering to the subject an anti-CTLA-4 antibody or an antigen-binding fragment thereof at a flat dose of between 650 mg to 850 mg.

Item 28. The method according to item 26, wherein the anti-CTLA-4 antibody or antigen-binding fragment thereof is administered at a dose of 10 mg/kg.

Item 29. The method according to item 27, wherein the anti-CTLA-4 antibody or antigen-binding fragment thereof is administered at a dose of 750 mg.

Item 30. The method according to any one of items 26 to 29, wherein the anti-CTLA-4 antibody or antigen-binding fragment thereof is tremelimumab.

Item 31. The method according to any one of items 26 to 30, wherein the anti-CTLA-4 antibody or the antigen-binding fragment thereof is administered every four weeks.

Item 32. The method according to any one of items 26 to 30, wherein the anti-CTLA-4 antibody or the antigen-binding fragment thereof is administered every twelve weeks.

Item 33. The method according to any one of items 26 to 30, further comprising administering transarterial chemoembolization (TACE).

Item 34. The method according to any one of items 28 to 33, wherein the tumor is a hepatocellular carcinoma (HCC).

Item 35. The method according to item 27, wherein the anti-CTLA-4 antibody or antigen-binding fragment thereof is tremelimumab administered at a dose of 750 mg every four weeks for seven doses followed by administration of tremelimumab at a dose of 750 mg every twelve weeks.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Durvalumab Administered as Monotherapy or Durvalumab in Combination with Tremelimumab in Subjects with Advanced Hepatocellular Carcinoma

The study disclosed below is a multicenter, open-label, stratified, study designed to evaluate the safety, tolerability, and clinical activity of durvalumab administered as monotherapy, and durvalumab in combination with tremelimumab, in subjects with advanced HCC (clintrials.gov identifier no. NCT03298451; HIMALAYA).

Subjects were male or female, ≥18 years of age (all countries except Japan) or ≥20 years of age (Japan only), with a diagnosis of advanced HCC confirmed pathologically or by noninvasive imaging methods and preserved liver function (Child-Pugh Score class A). Subjects were immunotherapy-naive and had either progressed on, are intolerant to, or had refused treatment with sorafenib or other VEGFR TKI.

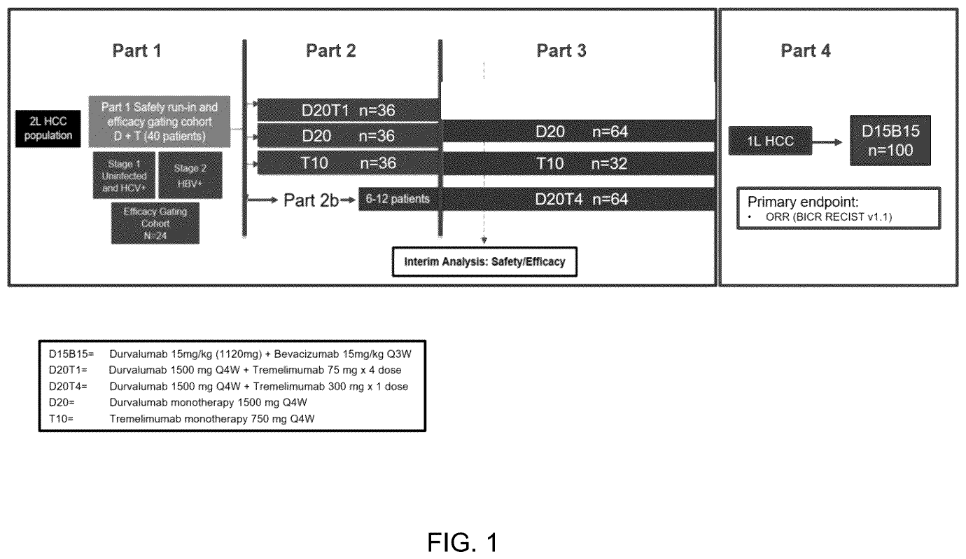

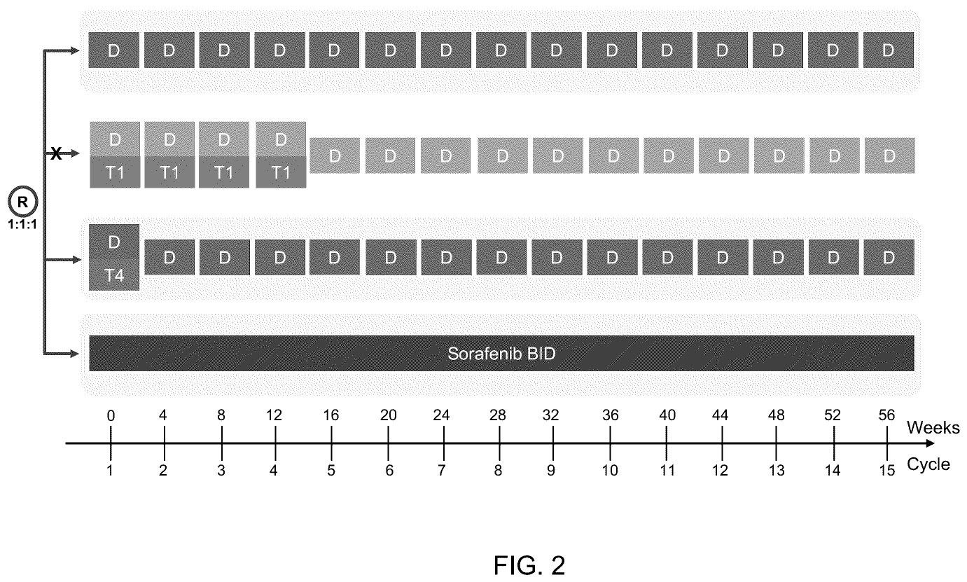

FIG. 1 shows the overall dosing schema of the study. FIG. 2 shows arms of the study as will be described in detail below. Patients were treated with durvalumab or tremelimumab as monotherapy or in combination and were given either weight-based on flat dosing regimens, depending on when they were enrolled in the study.

Part 1A: Safety Run-In with Durvalumab and Tremelimumab Combination Therapy

As shown in FIG. 1, subjects with advanced uninfected or HCV+ HCC were enrolled in Part 1A, Stage 1, for a safety run-in using a risk-based staggered approach. Subjects were administered the durvalumab 20 mg/kg and tremelimumab 1 mg/kg combination therapy every 4 weeks (Q4W) for 4 doses followed by durvalumab 20 mg/kg monotherapy Q4W until confirmed progressive disease (PD) or any other discontinuation criteria was met.

In stage 2, enrollment of additional subjects with advanced HBV+ HCC was started after the first subjects in stage 1 had been observed for at least 4 weeks. Subjects were administered durvalumab 20 mg/kg and tremelimumab 1 mg/kg combination therapy Q4W for 4 doses followed by durvalumab 20 mg/kg monotherapy Q4W until confirmed PD or any other discontinuation criteria was met.

### Part 1B: Efficacy Gating Cohort for Durvalumab and Tremelimumab Combination Therapy

Immunotherapy-naive subjects with advanced HCC who had progressed on, are intolerant of, or refused sorafenib-based therapy were enrolled in Part 1B for efficacy gating. Subjects (uninfected, HBV infected, or HCV infected) were enrolled in an efficacy gating cohort to determine if there was sufficient evidence of clinical activity to warrant opening enrollment to Part 2. Subjects were administered the durvalumab 20 mg/kg and tremelimumab 1 mg/kg combination therapy Q4W for 4 doses followed by durvalumab 20 mg/kg monotherapy Q4W until confirmed PD or any other discontinuation criteria was met. The first durvalumab monotherapy dose at 20 mg/kg Q4W was given 4 weeks after the final dose of durvalumab and tremelimumab combination therapy (see FIG. 2).

### Part 2A: Randomized Arms Evaluating Durvalumab and Tremelimumab Combination Therapy, Durvalumab Monotherapy, and Tremelimumab Monotherapy

As shown in FIG. 1, immunotherapy-naive subjects with advanced HCC who have progressed on, are intolerant of, or refused sorafenib-based therapy, were stratified based on viral status (uninfected, HCV infected, or HBV infected) and PD-L1 expression (positive, negative, or non-evaluable) in Part 2A to evaluate the durvalumab and tremelimumab as monotherapy or in combination. Subjects were randomized 1:1:1 within each stratum to 1 of the 3 treatment arms with approximately 36 subjects (approximately 12 subjects/viral status type) per treatment arm (see FIG. 2):
Arm A: durvalumab 20 mg/kg and tremelimumab 1 mg/kg combination therapy Q4W for 4 doses followed by durvalumab 20 mg/kg monotherapy Q4W until confirmed PD or any other discontinuation criteria was met. The first durvalumab monotherapy dose at 20 mg/kg Q4W was given 4 weeks after the final dose of durvalumab and tremelimumab combination therapy.
Arm B: durvalumab 20 mg/kg monotherapy Q4W until confirmed PD or any other discontinuation criteria was met.
Arm C: Tremelimumab 10 mg/kg monotherapy Q4W for 7 doses followed by every 12 weeks (Q12W) until confirmed PD or any other discontinuation criteria was met.

All subjects in Part 1A, Part 1B, and Part 2A were evaluated for efficacy and their disease status primarily analyzed according to RECIST v1.1. All subjects were followed for survival until the end of study.

### Part 2B: Safety Run-In for Additional Treatment Regimen of Durvalumab and Tremelimumab Combination Therapy

As shown in FIG. 1, immunotherapy-naive subjects with advanced HCC who have progressed on, are intolerant to, or have refused sorafenib-based therapy were enrolled in Part 2B for a safety run-in. Ten subjects were enrolled into Arm D evaluating a single higher dose of tremelimumab in combination with durvalumab:
Arm D: durvalumab 1500 mg and tremelimumab 300 mg combination therapy for 1 dose followed by durvalumab 1500 mg monotherapy Q4W until confirmed PD or any other discontinuation criteria was met. The first durvalumab monotherapy dose at 1500 mg Q4W was given 4 weeks after the final dose of durvalumab and tremelimumab combination therapy.

A safety evaluation was performed once 6 safety-evaluable subjects completed 4 weeks of follow-up. A safety-evaluable subject was defined as a subject who received at least 1 dose of study drug and completed at least 4 weeks of follow-up or discontinued treatment prior to the completion of 4 weeks of follow-up due to an adverse event.

### Part 3: Randomized Arms Evaluating Durvalumab and Tremelimumab Combination Therapy, Durvalumab Monotherapy, and Tremelimumab Monotherapy

As shown in FIG. 1, immunotherapy-naive subjects with advanced HCC who had progressed on, are intolerant to, or refused sorafenib or another approved VEGFR TKI-based therapy were stratified based on viral status (uninfected, HCV infected, or HBV infected) and sorafenib/VEGFR TKI treatment in Part 3 (sorafenib/VEGFR TKI refusers versus others). Subjects were randomized at a ratio of 2:1:2 into 1 of up to 3 treatment arms:
Arm A: durvalumab 1500 mg and tremelimumab 75 mg combination therapy Q4W for 4 doses followed by durvalumab 1500 mg monotherapy Q4W until confirmed PD or any other discontinuation criteria was met. The first durvalumab monotherapy dose at 1500 mg Q4W was given 4 weeks after the final dose of durvalumab and tremelimumab combination therapy.
Arm B: durvalumab 1500 mg monotherapy Q4W until confirmed PD or any other discontinuation criteria was met.
Arm C: Tremelimumab 750 mg monotherapy Q4W for 7 doses followed by Q12W until confirmed PD or any other discontinuation was met.
Arm D: durvalumab 1500 mg and tremelimumab 300 mg combination therapy for 1 dose followed by durvalumab 1500 mg monotherapy Q4W until confirmed PD or any other discontinuation criteria was met. The first durvalumab monotherapy dose at 1500 mg Q4W was given 4 weeks after the final dose of durvalumab and tremelimumab combination therapy.

All subjects in Part 3 were evaluated for safety and efficacy, and their disease status was primarily analyzed according to RECIST v1.1.

Primary analysis of all tumor assessment-related endpoints was based on BICR assessments according to RECIST v1.1.

Evaluation of target lesions were classified as follows:
Complete Response—Disappearance of all target lesions. Any pathological lymph nodes (whether target or non-target) must have reduction in short axis to <10 mm (the sum may not be “0” if there are target nodes).
Partial Response—At least a 30% decrease in the sum of the diameters of target lesions, taking as reference the baseline sum diameters.
Progressive Disease—At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: The appearance of one or more new lesions may be considered progression.)
Stable Disease—Neither sufficient shrinkage to qualify for PR nor sufficient increase to qualify for PD, taking as reference the smallest sum of diameters while on study.

Evaluation of non-target lesions will be classified as follows:
Complete Response—Disappearance of all non-target lesions and normalization of tumor marker level. All lymph nodes must be non-pathological in size (<10 mm in short axis).
Non-complete Response/Non-progressive Disease—Persistence of 1 or more non-target lesion(s) and/or maintenance of tumor marker level above the normal limits.
Progressive Disease—Unequivocal progression of existing non-target lesions will be defined as the overall level of substantial worsening in non-target disease such that, even in presence of SD or PR in target disease, the overall tumor burden has increased sufficiently to merit discontinuation of therapy. In the absence of measurable disease, change in nonmeasurable disease comparable in magnitude to the increase that would be required to declare PD for measurable disease. Examples include an increase in a pleural effusion from “trace” to “large,” an increase in lymphangitic disease from localized to widespread.

### Example 2: A Randomized, Open-Label, Multi-Center Clinical Study of Durvalumab and Tremelimumab as First-Line Treatment in Patients with Advanced Hepatocellular Carcinoma

The study disclosed below is a randomized, open-label, multi-center, global, clinical study to assess the efficacy and safety of durvalumab monotherapy and durvalumab plus tremelimumab combination therapy versus sorafenib in the treatment of immune checkpoint inhibitor-naïve patients with HCC who progressed on, were intolerant to, or refused sorafenib. In the foregoing disclosure, a flat-dose regimen of 1500 mg (approximately equivalent to 20 mg/kg) of durvalumab plus 300 mg (equivalent to 4 mg/kg) of tremelimumab was used in this study.

The study population included patients 18 years of age or older with advanced HCC, Barcelona Clinic Liver Cancer Stage B (not eligible for locoregional therapy) or stage C, and Child-Pugh A classification liver disease. Patients must not have received any prior systemic therapy for HCC. Patients were stratified according to macrovascular invasion (yes versus no), etiology of liver disease (hepatitis B virus [confirmed HBV] versus hepatitis C virus [confirmed HCV] versus others), and performance status (Eastern Cooperative Oncology Group [ECOG] 0 versus 1).

### Methods:

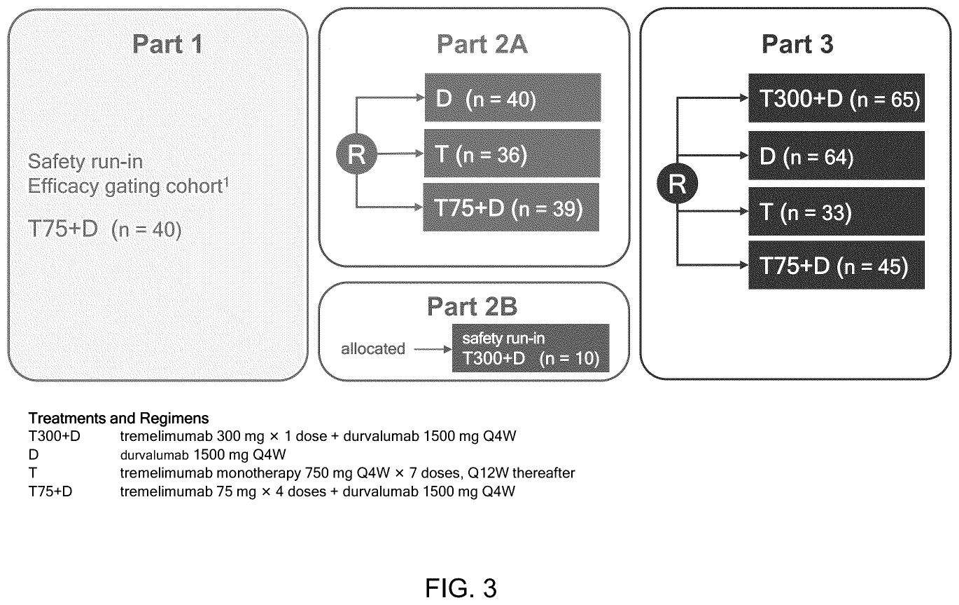

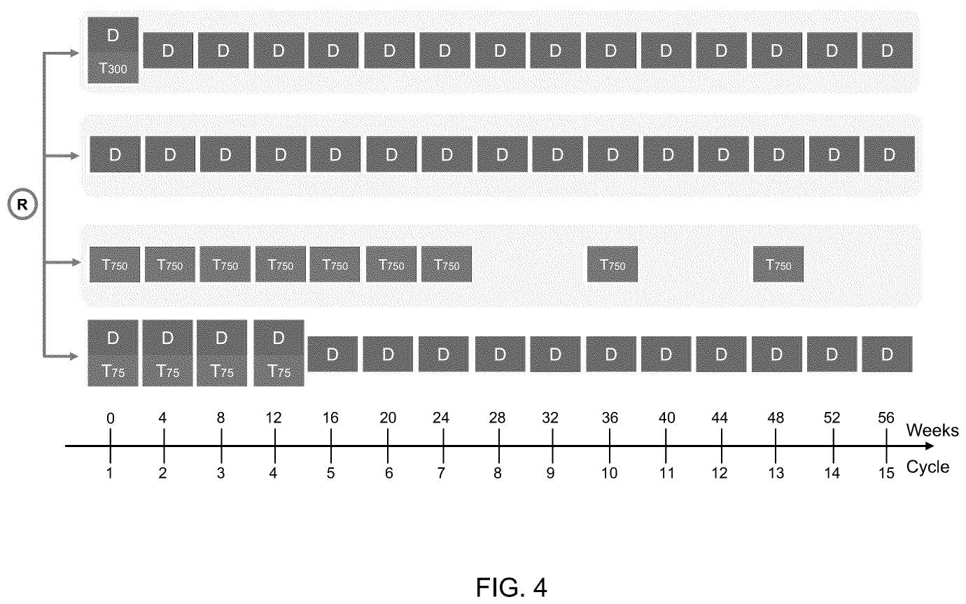

A total of 332 patients were enrolled (T300+D, n=75; durvalumab, n=104; tremelimumab, n=69; T75+D, n=84). Patients were randomized in an equal ratio to groups treated with durvalumab 1500 mg monotherapy (D), tremelimumab 750 mg monotherapy (T), combination therapy with durvalumab 1500 mg plus tremelimumab 75 mg×4 doses (T75+D) followed by durvalumab Q4W, combination therapy with durvalumab 1500 mg plus tremelimumab 300 mg×1 dose (T 300+D) followed by durvalumab Q4W. Upon closure to further enrollment into the T75+D arm, patients were randomized in an equal ratio to groups treated with D, T300+D, and Sorafenib. Durvalumab and tremelimumab were administered via intravenous (IV) infusion every 4 weeks (Q4W). Sorafenib was administered orally BID (see FIGS. 3 and 4).

### Tremelimumab 750 mg Monotherapy

Tremelimumab 750 mg via IV infusion Q4W starting at Week 0 for seven doses and then Q12W until confirmed PD, unacceptable toxicity, or any discontinuation criterion was met. (Note: If a patient's weight decreased to ≤30 kg, the patient received weight-based dosing of tremelimumab 10 mg/kg Q4W, until the weight increased to >30 kg, at which point the patient received the flat dose of tremelimumab 750 mg Q4W).

### Durvalumab 1500 mg Monotherapy

Durvalumab 1500 mg via IV infusion Q4W starting at Week 0 until confirmed PD, unacceptable toxicity, or any discontinuation criterion was met. (Note: If a patient's weight decreased to ≤30 kg, the patient received weight-based dosing of durvalumab 20 mg/kg Q4W, until the weight increased to >30 kg, at which point the patient received the flat dose of durvalumab 1500 mg Q4W.)

Durvalumab 1500 mg plus tremelimumab 75 mg×4 doses combination therapy (Arm B)
Durvalumab 1500 mg plus tremelimumab 75 mg×4 doses starting at Week 0, followed by durvalumab 1500 mg monotherapy Q4W starting 4 weeks after the final infusion of the combination therapy until confirmed PD, unacceptable toxicity, or any discontinuation criteria was met. (Note: If a patient's weight decreased to 30 kg or below (≤30 kg), the patient received weight-based dosing of durvalumab 20 mg/kg Q4W and tremelimumab 1 mg/kg Q4W, until the weight increased to >30 kg, at which point the patient received the original assigned flat dose of durvalumab 1500 mg Q4W with or without tremelimumab 75 mg Q4W.)

Durvalumab 1500 mg plus tremelimumab 300 mg×1 dose combination therapy (Arm C)
Durvalumab 1500 mg plus tremelimumab 300 mg×1 dose starting at Week 0, followed by durvalumab 1500 mg monotherapy Q4W starting 4 weeks after the first and final infusion of the combination therapy until confirmed PD, unacceptable toxicity, or any discontinuation criteria was met. (Note: If a patient's weight decreased to 30 kg or below (≤30 kg), the patient received weight-based dosing of durvalumab 20 mg/kg Q4W and tremelimumab 4 mg/kg×1 dose after until the weight increased to above 30 kg (>30 kg), at which point the patient received the original flat dose of durvalumab 1500 mg Q4W with or without tremelimumab 300 mg.)

Sorafenib 400 mg BID therapy (Arm D)
Sorafenib 400 mg (2×200-mg tablets) orally BID, until confirmed PD, unacceptable toxicity, or any discontinuation criteria was met.

### Results:

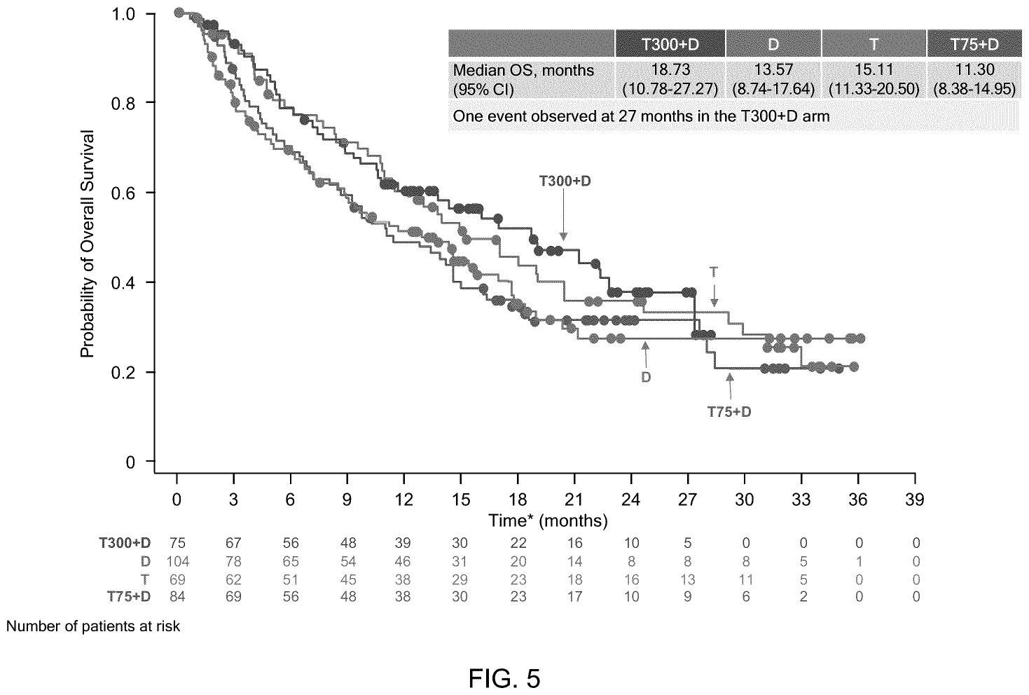

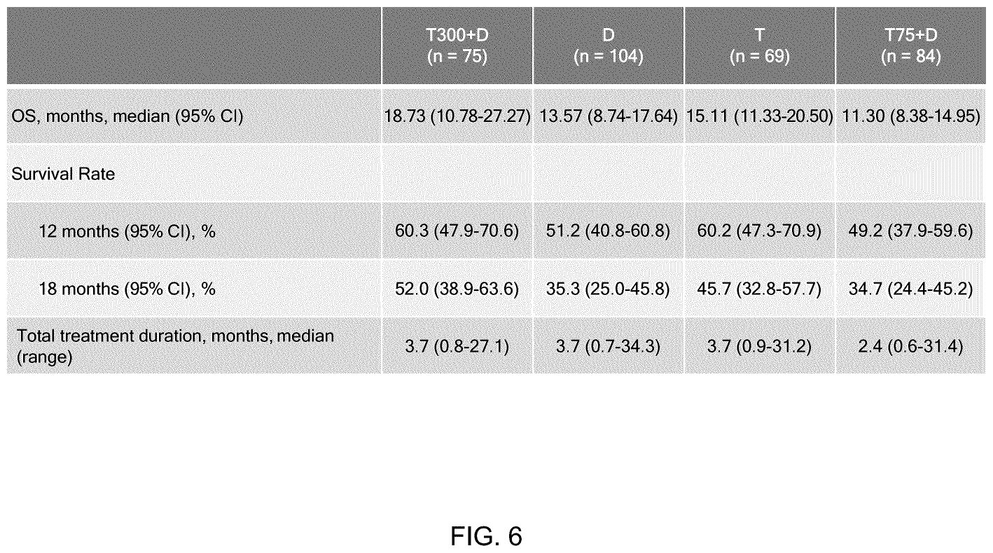

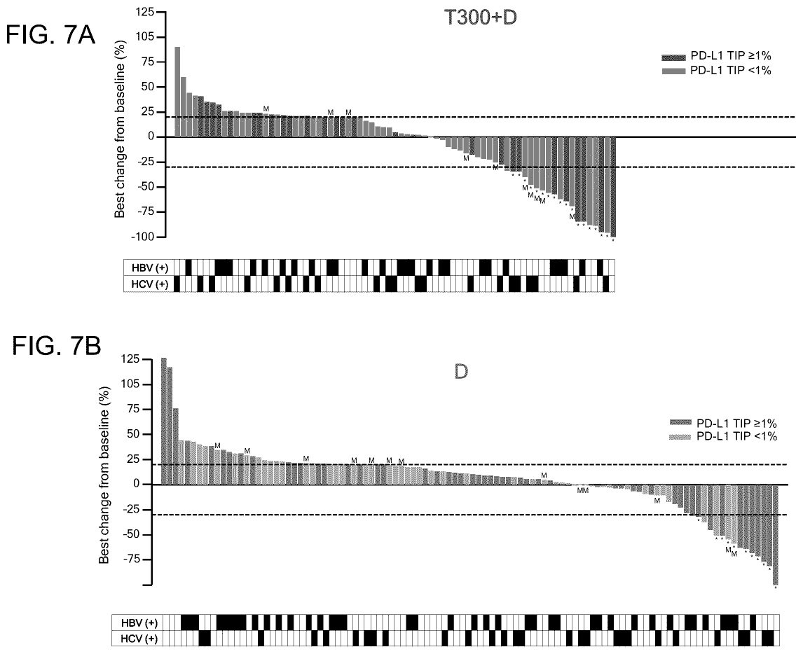

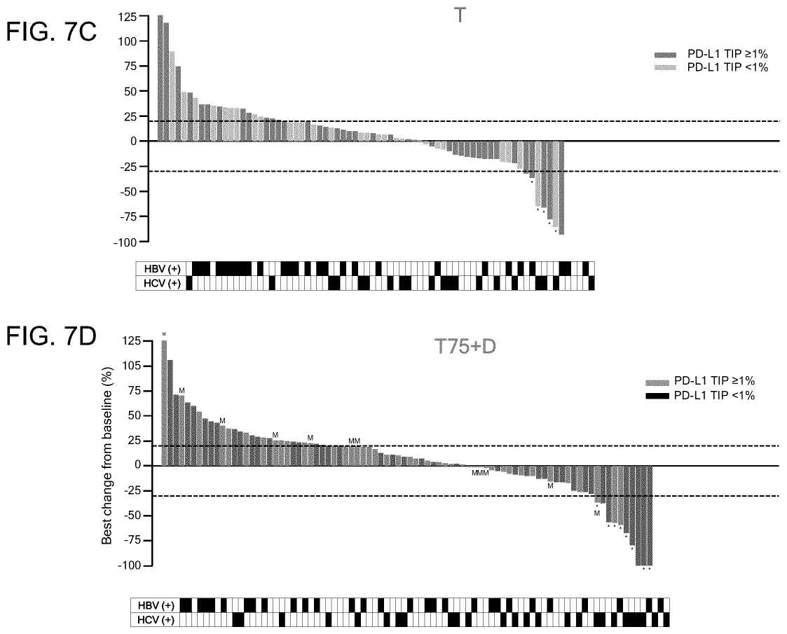

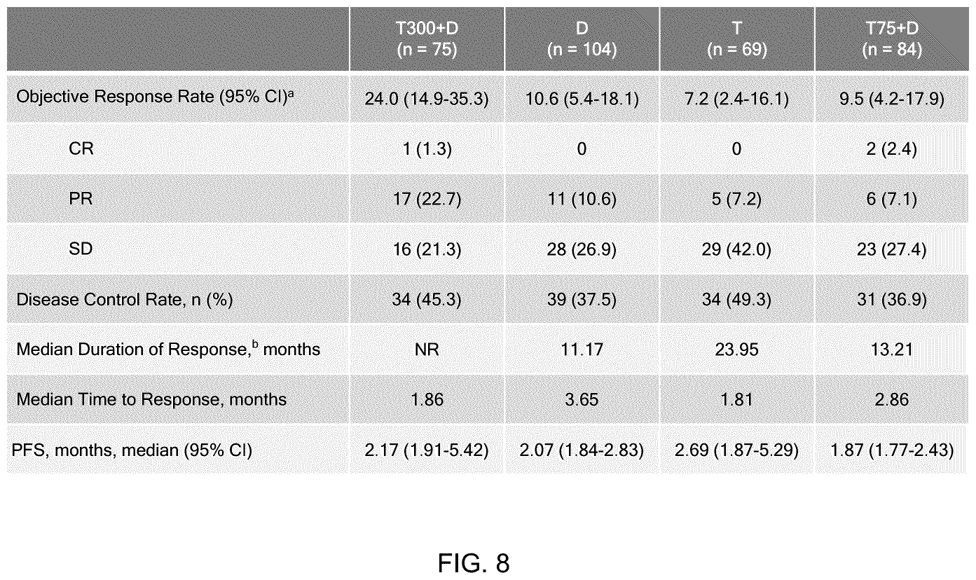

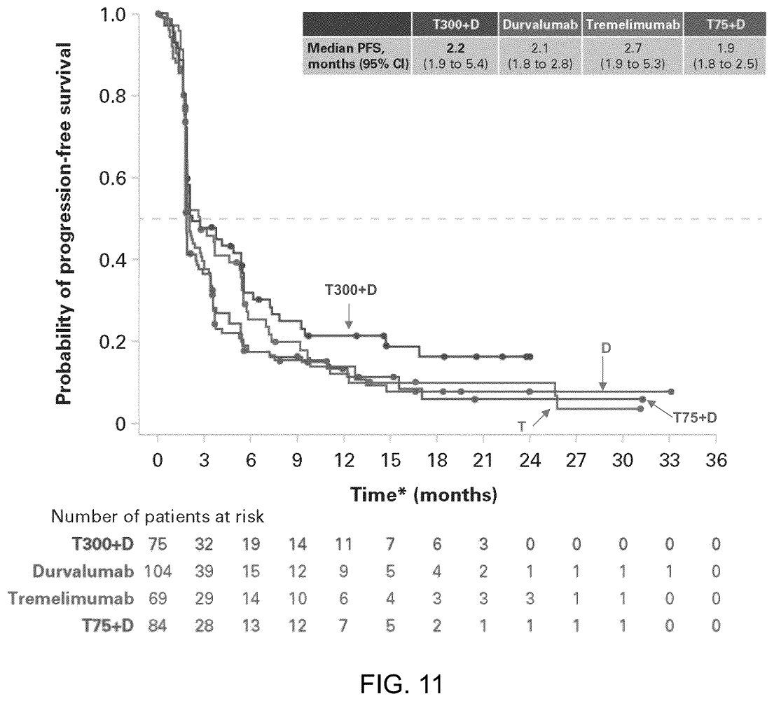

No new safety signals were identified beyond the established safety profile for each agent, alone or in combination. As shown in FIGS. 5 and 6, the patients in the T300+D arm had the highest confirmed ORR (24%) and longest OS (18.73 (10.78-27.27) months). Median PFS (95% CI) was 2.17 (1.91-5.42) months (T300+D) 2.07 (1.84-2.83) (durvalumab), 2.69 (1.87-5.29) (tremelimumab), and 1.87 (1.77-2.53) (T75+D) (FIG. 11). Across all arms, T300+D provided the best benefit-risk profile when compared with the other ICI regimens. Furthermore, these responses were observed regardless of PD-L1 expression level or viral status (see FIG. 7). FIG. 8 describes the secondary efficacy endpoints of the study.

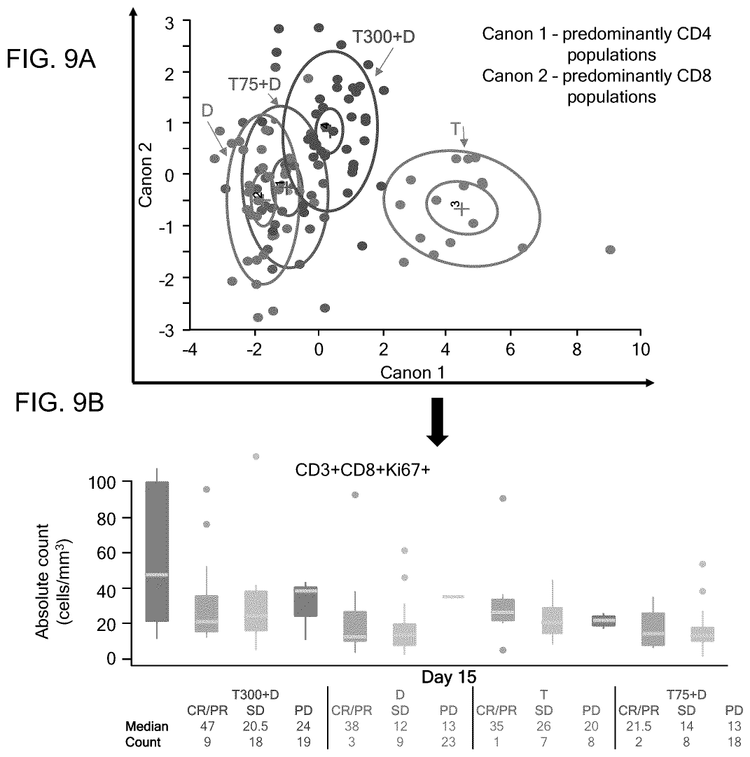

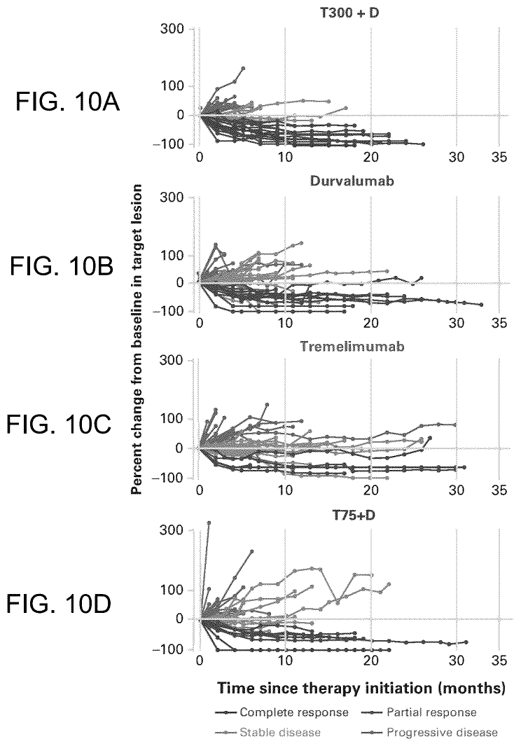

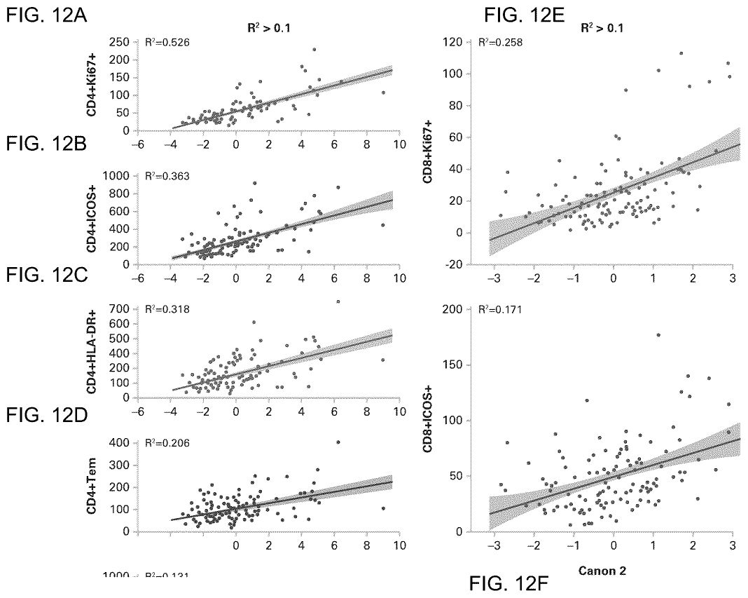

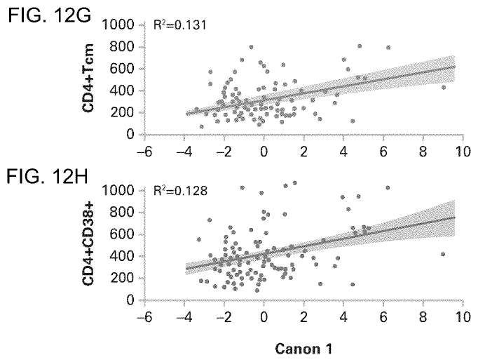

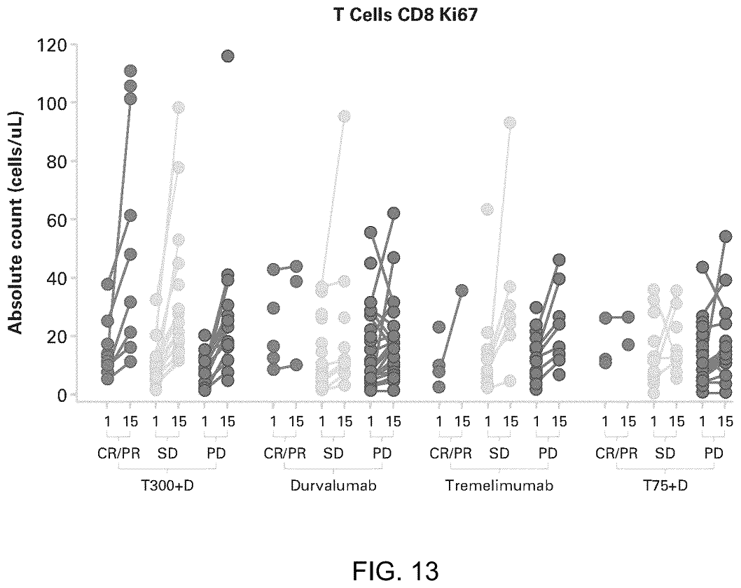

Furthermore, pharmacodynamic biomarker analysis showed patients with an overall response exhibited high cytotoxic (CD8) counts, indicating that T300+D drove a response-associated acute expansion of CD8 lymphocytes. This provided a unique proliferative T cell profile, suggesting additive biologic activity for the combination. Specifically, quadratic discriminant analysis of 26 lymphocyte population values on day 15 revealed patients were maximally discriminated by two discrete combinations of lymphocyte populations (canon-1 and canon-2) which were associated with CD4+ and CD8+ T-cells, respectively. Patients receiving T300+D exhibited the highest canon-2 scores (FIG. 9A). Linear regression analysis revealed that canon-2 was predominantly associated with elevations in the Ki67+ subset of CD8+ T cells (FIG. 12). Response was associated with an expansion of these CD8+Ki67+ lymphocytes occurring early during treatment (day 15). The highest median counts were observed with T300+D (FIGS. 9B and 13), consistent with the observation that T300+D yielded the highest ORR.

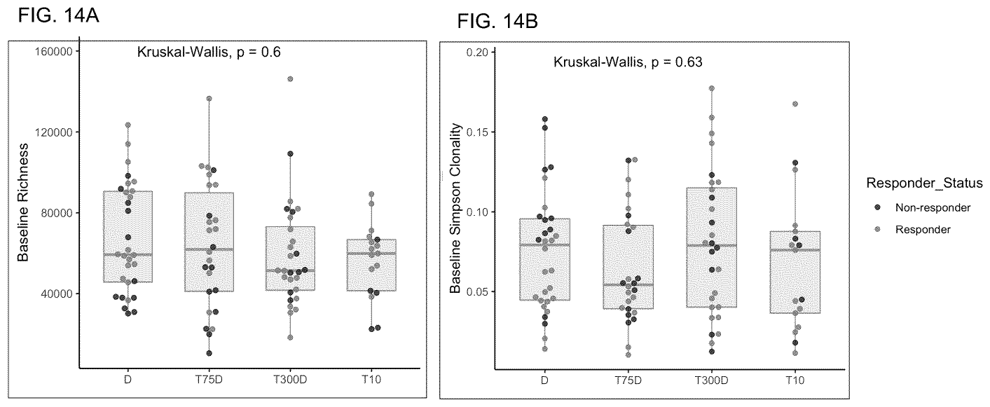

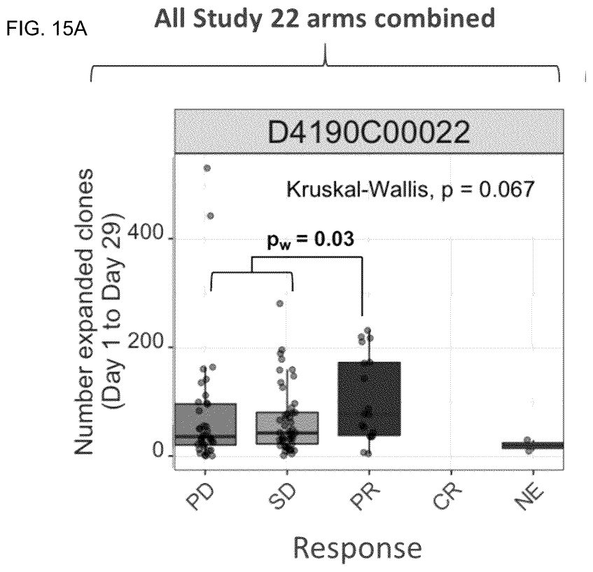

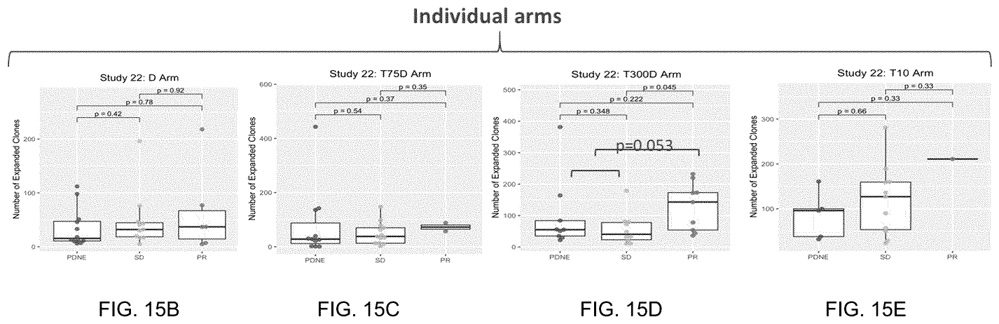

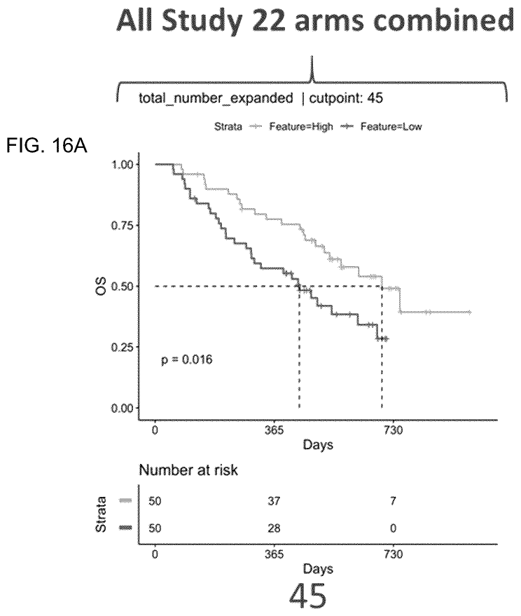

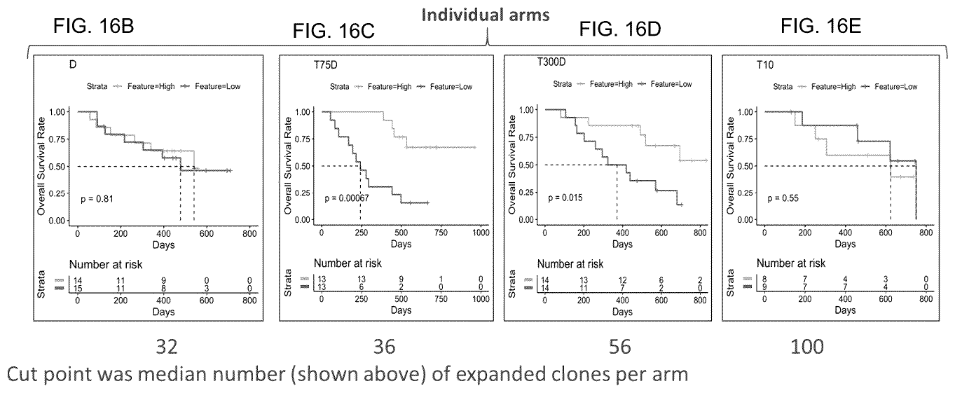

Molecular analysis of peripheral blood T cell receptors (TCRs) on day 29 during the first cycle of Q4W dosing demonstrated that the median increased T-cell clonal expansion at day 29 appeared to be T dose-dependent, with no significant difference between the D and T75+D arms. There were no significant differences in baseline richness or Simpson clonality of T-cells across arms (FIG. 14). Greater clonal expansion of T cells was associated with response, and appeared to be driven by higher doses of T. Specifically, across all arms, responders had a larger median number of expanded T-cell clones on Day 29 than non-responders (77.5 versus 40). Responders treated with T300D show significantly increased clonal expansion compared to non-responders. This trend was not seen with monotherapy D or the lower dose T combination (T75+D) (FIG. 15). Additionally, greater clonal expansion of T-cells was associated with better OS and seen in D+T combination arms (FIG. 16).

TABLE 1
Evaluable samples, TCR clonality, and clinical outcomes

| | T300 + D | D | T | T75 + D |
|---|---|---|---|---|
| | (n = 75) | (n = 104) | (n = 69) | (n = 84) |
| immunoSeq paired | 30 | 31 | 17 | 26 |
| samples, n | | | | |
| Dose of T before | 300 | 0 | 750 | 75 |
| Day 29, mg | | | | |
| ORR, % | 24.0 | 10.6 | 7.2 | 9.5 |
| Median (95% CI) | 18.7 | 13.6 | 15.1 | 11.3 |
| OS, mo | (10.8-27.3) | (8.7-17.6) | (11.3-20.5) | (8.4-15.0) |
| Median expanded | 56 (36, 84) | 32 (13, 46) | 100 (50, 160) | 36 (22, 70) |
| T cell clones at | | | | |
| Day 29, n | | | | |

Finally, an exposure-response model was developed to describe the relationship between tremelimumab exposure and proliferating CD8+ Ki67+ T cells in patients with uHCC. Relationships between tremelimumab trough concentration after the first dose (Cmin) and the maximum change from baseline (CFB) for CD8+ Ki67+ T cell counts were evaluated using linear and non-linear regression models. Covariate effects were evaluated on the model intercept and the drug effect (Emax) using a stepwise search approach. The relationship between CD8+Ki67+ T cell count CFB and tremelimumab trough concentration was best described by an Emax model. The only statistically significant and well estimated covariate effect was the baseline CD8+ Ki67+ T cell count on Emax. The maximum effect was higher in patients with lower baseline CD8+ Ki67+ T cell counts and lower in patients with higher baseline CD8+ Ki67+ T cell counts. At the 10th, median, and 90th percentiles of baseline CD8+(3, 9, and 25 cells/uL), the maximum effect is 593%, 341%, and 77.9% change from baseline, respectively. The estimated half-maximal effective concentration (EC50) was 5.24 ug/mL, which is well below the median Cmin for 300 mg tremelimumab of 12.9 ug/mL. The exposure-response analysis suggests that the saturable relationships observed in proliferating CD8+Ki67+ T cells is close to Emax for the T300+D regimen.

The results described above show that a single, priming dose of tremelimumab combined with monthly durvalumab resulted in clinically-meaningful outcomes in HCC populations.

1.-35. (canceled)

36. A method of extending progression-free survival (PFS) in a subject with cancer comprising treating the subject with
(a) a therapeutically effective amount of an anti-PD-L1 antibody or antigen-binding fragment thereof and a transarterial chemoembolization (TACE) procedure; and
(b) a therapeutically effective amount of an anti-PD-L1 antibody or antigen-binding fragment thereof, and a therapeutically effective amount of an anti-vascular endothelial growth factor (anti-VEGF) antibody or antigen-binding fragment thereof.

37. A method of extending overall survival (OS) in a subject with cancer comprising treating the subject with
(a) a therapeutically effective amount of an anti-PD-L1 antibody or antigen-binding fragment thereof and a transarterial chemoembolization (TACE) procedure; and
(b) a therapeutically effective amount of an anti-PD-L1 antibody or antigen-binding fragment thereof, and a therapeutically effective amount of an anti-vascular endothelial growth factor (anti-VEGF) antibody or antigen-binding fragment thereof.

38. The method of claim 36, wherein the therapeutically effective amount of an anti-PD-L1 antibody or antigen-binding fragment thereof of (a) is administered at a dose of 1500 mg.

39. The method of claim 36, wherein the therapeutically effective amount of an anti-PD-L1 antibody or antigen-binding fragment thereof of (b) is administered at a dose of 1120 mg.

40. The method of claim 36, wherein the therapeutically effective amount of an anti-PD-L1 antibody or antigen-binding fragment thereof is administered intravenously.

41. The method of claim 36, wherein the therapeutically effective amount of an anti-PD-L1 antibody or antigen-binding fragment thereof is durvalumab.

42. The method of claim 36, wherein the therapeutically effective amount of an anti-VEGF antibody or antigen-binding fragment thereof is bevacizumab.

43. The method of claim 36, wherein in step (a) the therapeutically effective amount of an anti-PD-L1 antibody or antigen-binding fragment thereof and the transarterial chemoembolization (TACE) procedure, are administered in combination.

44. The method of claim 36, wherein the transarterial chemoembolization (TACE) procedure occurs every 4 weeks.

45. The method of claim 36, wherein four transarterial chemoembolization (TACE) procedures occur within 16 weeks of initiating therapy.

46. The method of claim 36, wherein the administration of the therapeutically effective amount of an anti-PD-L1 antibody or antigen-binding fragment thereof and the TACE procedure occur on different days, and wherein the administration of the therapeutically effective amount an anti-PD-L1 antibody or antigen-binding fragment thereof and the TACE procedure are separated by at least seven days.

47. The method of claim 36, wherein the administration of the therapeutically effective amount of an anti-PD-L1 antibody or antigen-binding fragment thereof and the therapeutically effective amount of an anti-vascular endothelial growth factor (anti-VEGF) antibody or antigen-binding fragment thereof and the TACE procedure are separated by at least fourteen days.

48. The method of claim 36, wherein the cancer comprises hepatocellular cancer (HCC).

49. The method of claim 36, wherein the subject with cancer was previously unresponsive to a curative therapy.

50. The method of claim 49, wherein the curative therapy is surgical resection, transplantation, or ablation.

51. The method of claim 36, wherein step (b) occurs after completion of step (a).

52. The method of claim 36, wherein in step (a) the therapeutically effective amount of an anti-PD-L1 antibody or antigen-binding fragment thereof is administered every four weeks.

53. The method of claim 36, wherein in step (b) the therapeutically effective amount of an anti-PD-L1 antibody or antigen-binding fragment thereof is administered every three weeks.

54. The method of claim 36, wherein in step (b) the therapeutically effective amount of an anti-PD-L1 antibody or antigen-binding fragment thereof and the anti-VEGF antibody or antigen-binding fragment thereof are administered every two to four weeks.

55. A method of treating or preventing a disease or disorder comprising administering to a subject in need thereof a therapeutically effective amount of:
(a) a therapeutically effective amount of an anti-PD-L1 antibody or antigen-binding fragment thereof is administered every four weeks and a transarterial chemoembolization (TACE) procedure; and
(b) a therapeutically effective amount of an anti-PD-L1 antibody or antigen-binding fragment thereof is administered every three weeks, and a therapeutically effective amount of an anti-VEGF antibody or antigen-binding fragment thereof is administered every two to four weeks.
